(19) United States
(12) Patent Application Publication
(10) Pub. No.: US 2014/0316142 A1
(43) Pub. Date: Oct. 23, 2014

(54) PROCESS FOR THE PREPARATION OF ANGIOTENSIN II ANTAGONISTS AND INTERMEDIATES THEREOF

(71) Applicant: ALKEM LABORATORIES LTD., Mumbai (IN)

(72) Inventors: Mahendranath Narasimha Rao Rao, Bangalore (IN); Lokesh Ravilla, Chittoor (IN); Shridhara Kanakamajalu, Bangalore (IN); Sundarraja Kothapalli Rao, Bangalore (IN); Kuppuswamy Nagarajan, Bangalore (IN)

(73) Assignee: ALKEM LABORATORIES LTD., Mumbai (IN)

(21) Appl. No.: 14/346,831

(22) PCT Filed: Sep. 18, 2012
(86) PCT No.: PCT/IN2012/000622

§ 371 Date: Mar. 24, 2014

(30) Foreign Application Priority Data
Sep. 27, 2011 (IN) 2720/MUM/2011

(51) Int. Cl. C07D 257/04

(52) U.S. Cl. CPC C07D 257/04
USPC 548/253

(57) ABSTRACT

The present invention relates to an improved process for the preparation of angiotensin receptor antagonists and intermediates thereof. Particularly the present invention relates to an improved process for the preparation of N-(1-oxopentyl)-N-[[2′-(1H-tetrazol-5-yl)[1,1′-biphenyl]-4-yl]methyl]-L-valine of Formula 1

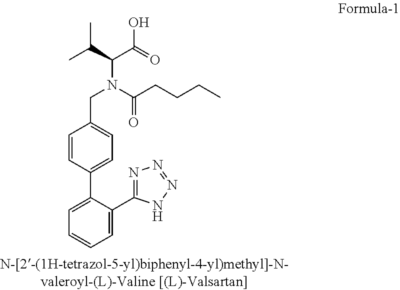 [SMILES: CCCCC(=O)N(Cc1ccc(-c2ccccc2-c2nnn[nH]2)cc1)[C@H](C(=O)O)C(C)C]

via a phosphite salt of Formula-4.H₃PO₃

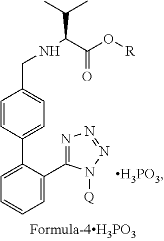

preferably a phosphite salt of Formula-4′.H₃PO₃

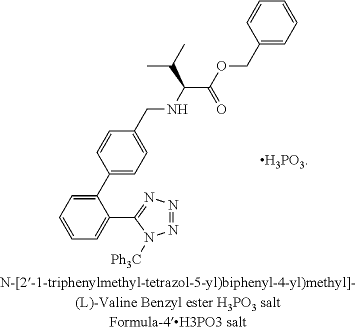

### FIELD OF THE INVENTION

The present invention relates to an improved process for the preparation of angiotensin receptor antagonists and intermediates thereof. Particularly the present invention relates to an improved process for the preparation of N-(1-oxopentyl)-N-[[2′-(1H-tetrazol-5-yl)[1,1′-biphenyl]-4-yl]methyl]-L-valine of Formula 1.

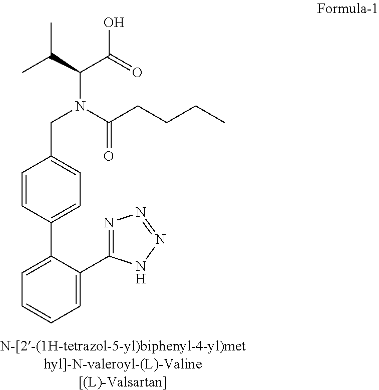 [SMILES: CCCCC(=O)N(Cc1ccc(-c2ccccc2-c2nnn[nH]2)cc1)[C@H](C(=O)O)C(C)C]

### BACKGROUND OF THE INVENTION

Angiotensin receptor antagonists are indicated for the treatment of hypertension. Angiotensin II formed from Angiotensin I receptor in a reaction catalyzed by angiotensin-converting enzyme (ACE, kininase II) is the principal suppressor agent and has effects that include vasoconstriction, cardiac stimulation, renal absorption of sodium and also stimulation of synthesis and release of aldosterone. Representative examples of angiotensin receptor antagonists include biphenyl compounds of the like of Valsartan, Losartan, Irbesartan, and Candesartan.

N-(1-Oxopentyl)-N-[[2′-(1H-tetrazol-5-yl)[1,1′-biphenyl]-4-yl]methyl]-L-valine, generically known as Valsartan, is a nonpeptide, orally active, and specific angiotensin II antagonist acting on the AT₁ receptor subtype. Valsartan is used for the treatment of hypertension and is marketed as the free acid under the name DIOVAN®.

U.S. Pat. No. 5,399,578 (herein referred to as US′578) assigned to Ciba-Geigy discloses Valsartan and its pharmaceutically acceptable salts. It describes three different processes for the preparation of Valsartan. One of these processes involves the reaction of 4-bromomethyl-2′-cyanobiphenyl (II) with L-valine methyl ester, followed by treatment with valeroyl chloride to produce 2-amino-N-[(2′-cyanobiphenyl-4-yl)methyl]-2-methyl-N-valeryl propionate (IV). Compound (IV) is treated with tri-n-butyl tin azide to give N-(1-oxopentyl)-N-[[2′-(1H-tetrazol-5-yl)[1,1′biphenyl]-4-yl]methyl-L-valine methyl ester (V), which is then hydrolyzed under alkaline condition to give finally Valsartan. The process is shown in Scheme-I below:

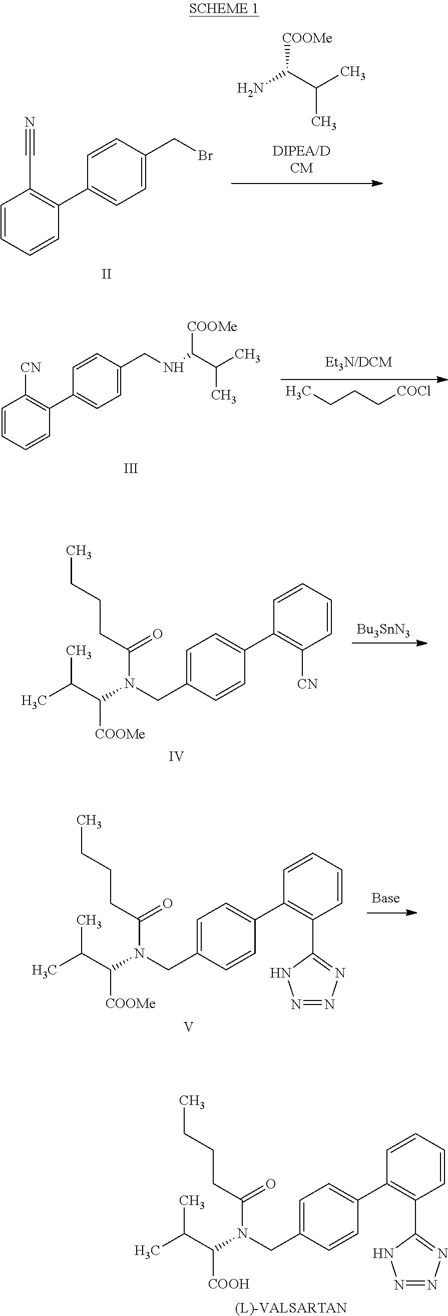

An alternative method of synthesis as taught by this patent involves using 2-cyano-4-formylbiphenyl instead of 4-bromomethyl-2′-cyanobiphenyl (II).

The above-mentioned methods suffer from serious disadvantages in that it involves the use of toxic tributyl tin azide to build the tetrazole ring and thus raises serious safety demands so as to prevent explosion hazards due to the formation of hydrogen azide during the reaction.

Another alternative method as taught by US′578 involves reaction of 4-bromomethyl-2′-(1-triphenylmethyltetrazol-5-yl)biphenyl (VI) with L-valine benzyl ester to produce N-[[2′-(1-triphenylmethyltetrazol-5-yl)biphenyl-4-yl]-methyl]-L-valine benzyl ester (VII), followed by treatment with valeroyl chloride to produce N-(1-oxopentyl)-N-[[2′-(1-triphenylmethyltetrazol-5-yl)[1,1′-biphenyl]-4-yl]-methyl]-L-valine benzyl ester (VIII). Compound (VIII) is deprotected under acidic conditions to produce N-(1-oxopentyl)-N-[[2′-(1H-tetrazol-5-yl)[1,1′-biphenyl]-4-yl]-methyl]-L-valine benzyl ester (IX), which is then hydrogenated in presence of Pd/C catalyst to give finally Valsartan. The process is shown in scheme II, below:

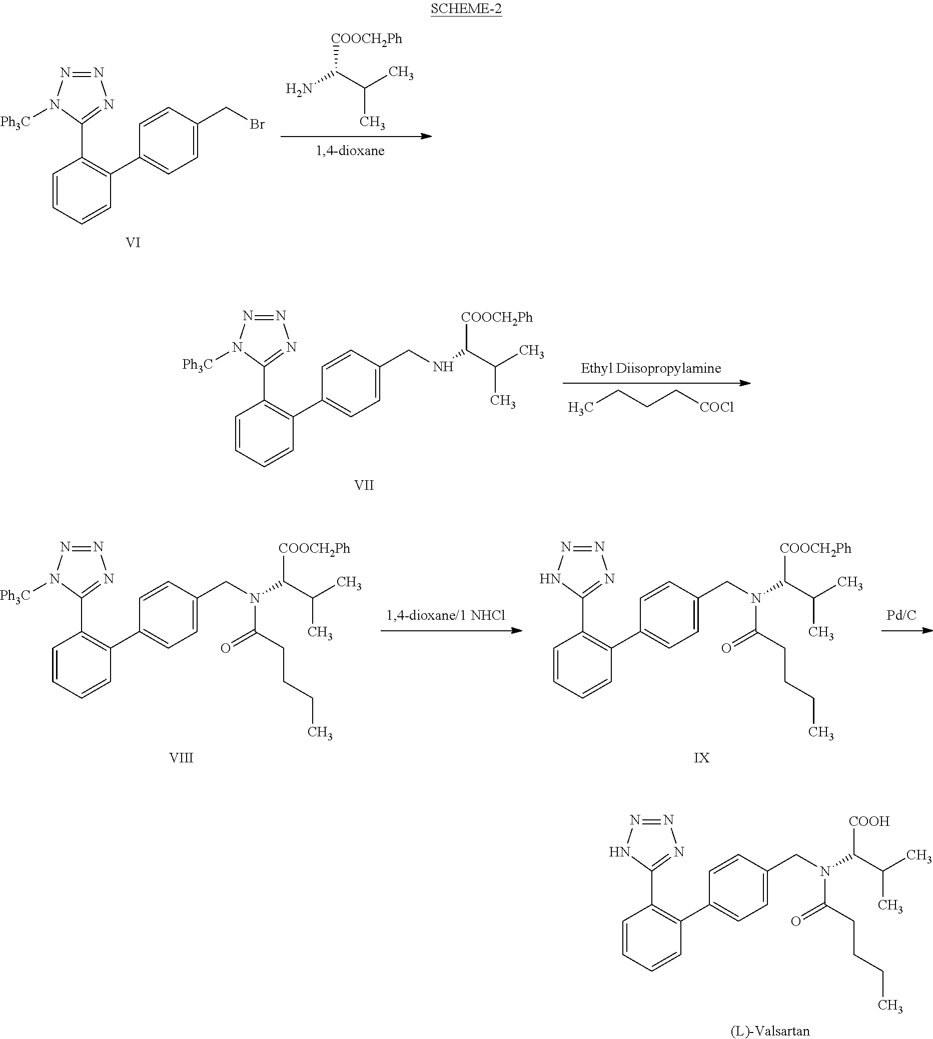

All the intermediates except the compound (IX) are oily substances, which cannot be crystallized. The final product is therefore, strongly contaminated with undesired compounds and requires repeated crystallization, resulting in a significant loss of yield.

An accurate reproduction of the processes as taught by Scheme II of US'578 by a person skilled in the art, is rather problematic, and also Example 55 of US'578 which documents Scheme II, is quite without data on weighed quantities in two steps, and in case of Example 55a, it is even referred to as analogous Example 57a, which does not relate to the given reaction at all. A replication of the process as taught by Example 55 by our inventors resulted in Valsartan of inferior quality and reduced yields and also required multiple crystallizations-thereby increasing time, cost and labour. The process as taught by US'578 is not practically feasible in an industrial scale.

WO 2004/101534 describes a variation to the above process of preparation of Valsartan, which involves isolation of N-[[2′-(1-triphenylmethyltetrazol-5-yl) biphenyl-4-yl]methyl]valine benzylester (VII) in the form of hydrochloride salt. The hydrochloride salt is further converted to Valsartan. However, a major disadvantage of this process is low yield and low purity of N-[[2′-(1-triphenylmethyltetrazol-5-yl)biphenyl-4-yl]methyl]valine benzyl ester (VII), which is the key intermediate in the preparation of Valsartan due to its high instability towards strong acidic conditions (hydrochloric acid) and its tendency to undergo hydrolysis to generate undesired impurities, which get carried forward as impurities in Valsartan (I). Removal of impurities in the final stage is not only tedious and time consuming, but also a costly process requiring large amounts of solvents for the repeated re-crystallization steps until the desired purity is attained.

US 20060281801 discloses yet another process for the preparation of valsartan. It provides a purification method for the removal of the organo tin impurity from benzyl valsartan by a process comprising the steps of crystallization of benzyl valsartan from a ternary solvent mixture comprising a hydrophilic organic solvent, a non-polar organic solvent, and water, Crystallization from a polar aprotic solvent, a non-polar organic solvent, or mixtures thereof. Preferably, the second crystallization solvent is a binary solvent mixture comprising a polar aprotic solvent and a non-polar organic solvent.

U.S. Pat. No. 7,199,144 (herein referred to as US '144) discloses yet another process for the preparation of valsartan and precursors thereof similar to a process as disclosed in US '578, but it provides alternative methods to remove residual solvent, particularly ethyl acetate (of which the permissible limit is less than 5000 ppm) from the final product by a process comprising the steps of trituration of the crude Valsaratan with water, performing a solvent exchange by contact of the solvate with humid gas in a fluidized bed, by harsh drying of valsartan at a temperature of about 5 to about 60 degree C. under a pressure of less than about 30 mm Hg. However the US'144 patent too is associated with the same drawback as that of US'578 in that it too involves the production of oily intermediates, unavoidable use of toxic azides and the like.

U.S. Pat. No. 7,741,507 (herein referred to as US'507) discloses yet another process for the preparation of valsartan via a novel intermediate of formula

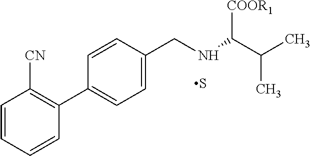

wherein S is an organic acid addition salt. This process too suffers from the drawback of unavoidable use of toxic azides and the like.

U.S. Pat. No. 7,880,015 (herein referred to as US'015) discloses a process for the preparation of valsartan which involves isolation of N-[[2′-(1-triphenylmethyltetrazol-5-yl)biphenyl-4-yl]methyl]valine methyl ester in the form of its oxalate acid addition salt

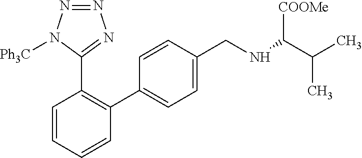

However the US'015 process would involve two de-protection steps-firstly-removal of the Trityl protecting group via acidic hydrolysis followed by de-esterification of the Methyl group by basic hydrolysis. The prior art process of removal of trityl group in acidic conditions with acids of the like of HCl, sulphuric acid, acetic acid resulted in an ‘oily’ product of low quality and purity. Also, subsequent hydrolysis of the ester in basic conditions with bases of the like of NaOH, LiOH, KOH, Ba (OH)₂, Ca(OH)₂ lead to racemisation which further necessitated an additional resolution step to afford (L) valsartan with significant loss of yield.

The inventors of the present invention have devised a novel strategy involving simultaneous de protection of both the trityl as well as the benzylic ester moieties via a single one-pot de-hydrogenation process-thereby saving time, cost and reduced reaction-time cycle.

US20110105763 discloses yet another process for the preparation of valsartan which involves isolation of N-[[2′-(1-triphenylmethyltetrazol-5-yl) biphenyl-4-yl]methyl]valine benzyl ester

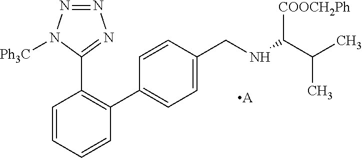

in the form of its organic carboxylic acid addition salt. However this application too follows the conventional two step de-protection procesS-firstly de-tritylation with a number of acid variants of the like of HCl, H₂SO₄, oxalic acid, p-toluene sulfonic acids and the like followed by de-benzylation.

The processes disclosed in the prior art are thus cumbersome and not feasible industrially. There is a need to develop a more efficient and economical synthetic route suitable for industrial scale-up.

The present inventors have devised an improved process for the preparation of valsartan primarily targeting on obtaining an ester of N-[[2′-(1-triphenylmethyltetrazol-5-yl) biphenyl-4-yl]methyl]valine of high purity. Surprisingly the present inventors have found that the above key starting material could be prepared by coupling a biphenyltetrazole methyl halide and L-valine benzyl ester in the absence of a base, wherein the L-valine benzyl ester itself acts as an in situ base that would quench the HX salt released during the reaction. An N-alkylation reaction in the absence of a base is not disclosed in the prior-art.

The present inventors have provided a solution to the problem associated with providing valsartan of high yield and purity in a simple and cost-effective manner by devising a strategy involving total absence of a base for the N-alkylation reaction involved in the synthesis of a key intermediate-N-[[2′-(1-triphenylmethyltetrazol-5-yl)biphenyl-4-yl]methyl]valine benzyl ester and its subsequent purification via its phosphorous acid addition salt. The prior art processes taught the N-alkylation of a Valine ester with a halo compound in the presence of an organic or inorganic base. The resultant N-alkylated intermediate so obtained by prior-art processes was always a gummy mass that required repeated solvent re-crystallizations, yet with a purity far lesser and quality inferior as compared to that obtained by a process of the present invention. In contrast, the N-alkylated stage-1 intermediate obtained by a process of the present invention was converted to its phosphorous acid addition salt with a purity as high as 97%. Further in the process of the present invention, the phosphite salt of stage-1 intermediate was either re-converted back to the free base with a base in a solvent or simply directly used in the second stage without requiring any further re-crystallisation or purification techniques unlike the prior art processes wherein repeated solvent re-crystallizations were a necessity to convert an impure and gummy stage-1 intermediate to acceptable purity levels fit enough to be taken for the next stage. Alternatively, process of the present invention facilitates almost 90% recovery of L-valine benzyl ester which can be re-used directly again for another batch of the same step. Thereby the process of the present invention not only avoids the unnecessary use of an external base for the N-alkylation reaction in Stage-1 by employing excess L-valine benzyl ester to perform a dual role of both as a reactant as well as an in situ base, but also succeeds in obtaining the N-alkylated stage-1 intermediate in high purity and yields without involving the cumbersome and repetitive solvent re-crystallisation techniques of the prior art. The novel process of the present invention is economically viable, reproducible, cost effective and industrially applicable.

### SUMMARY OF THE INVENTION

The objective of the present invention is to provide an improved process for the preparation of angiotensin II antagonists and intermediates thereof. Representative examples of angiotensin receptor antagonists include biphenyl compounds of the like of Valsartan, Losartan, Irbesartan, and Candesartan. Particularly, the invention relates to an improved process for the preparation of valsartan (I) and its pharmaceutically acceptable salts or intermediates thereof.

The process of the present invention is described in Scheme-3 below. Scheme-4 illustrates a preferred aspect of the present invention.

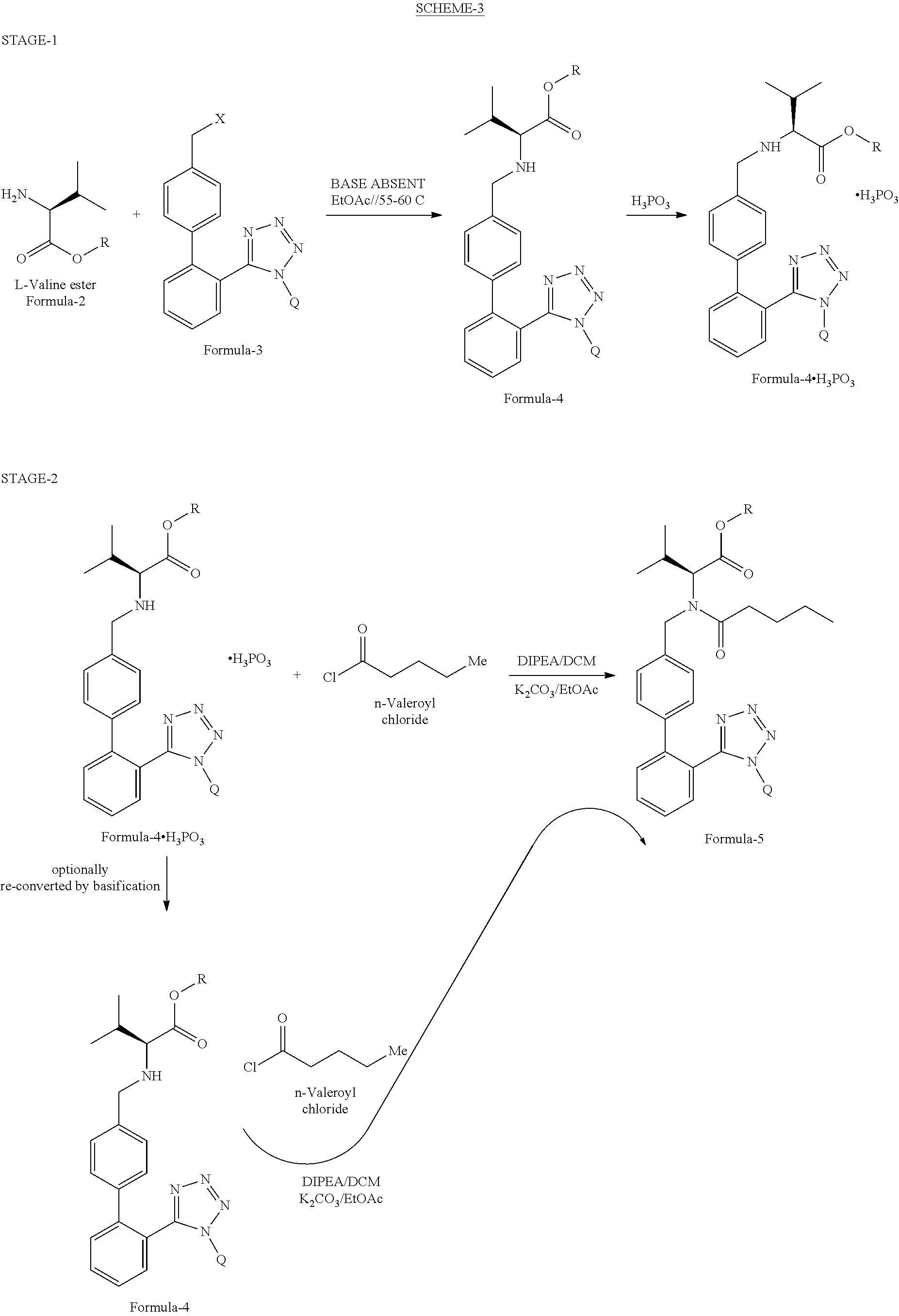

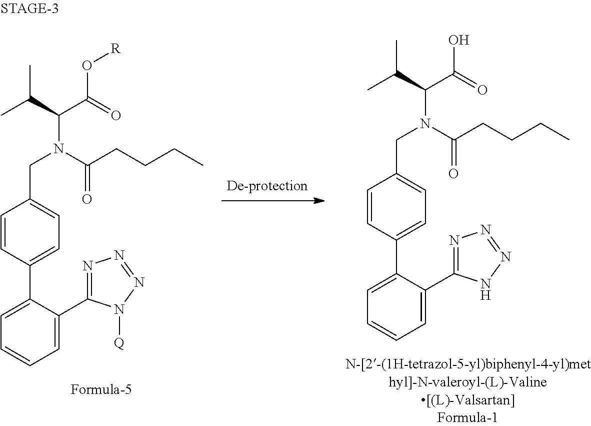

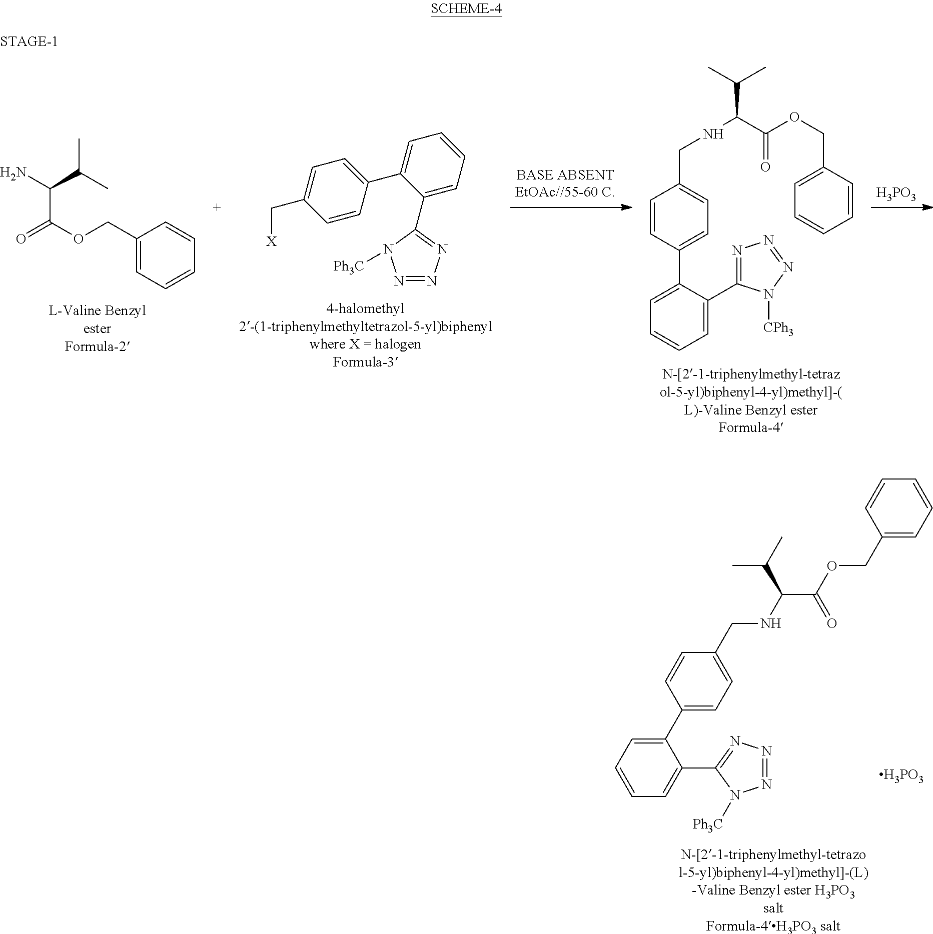

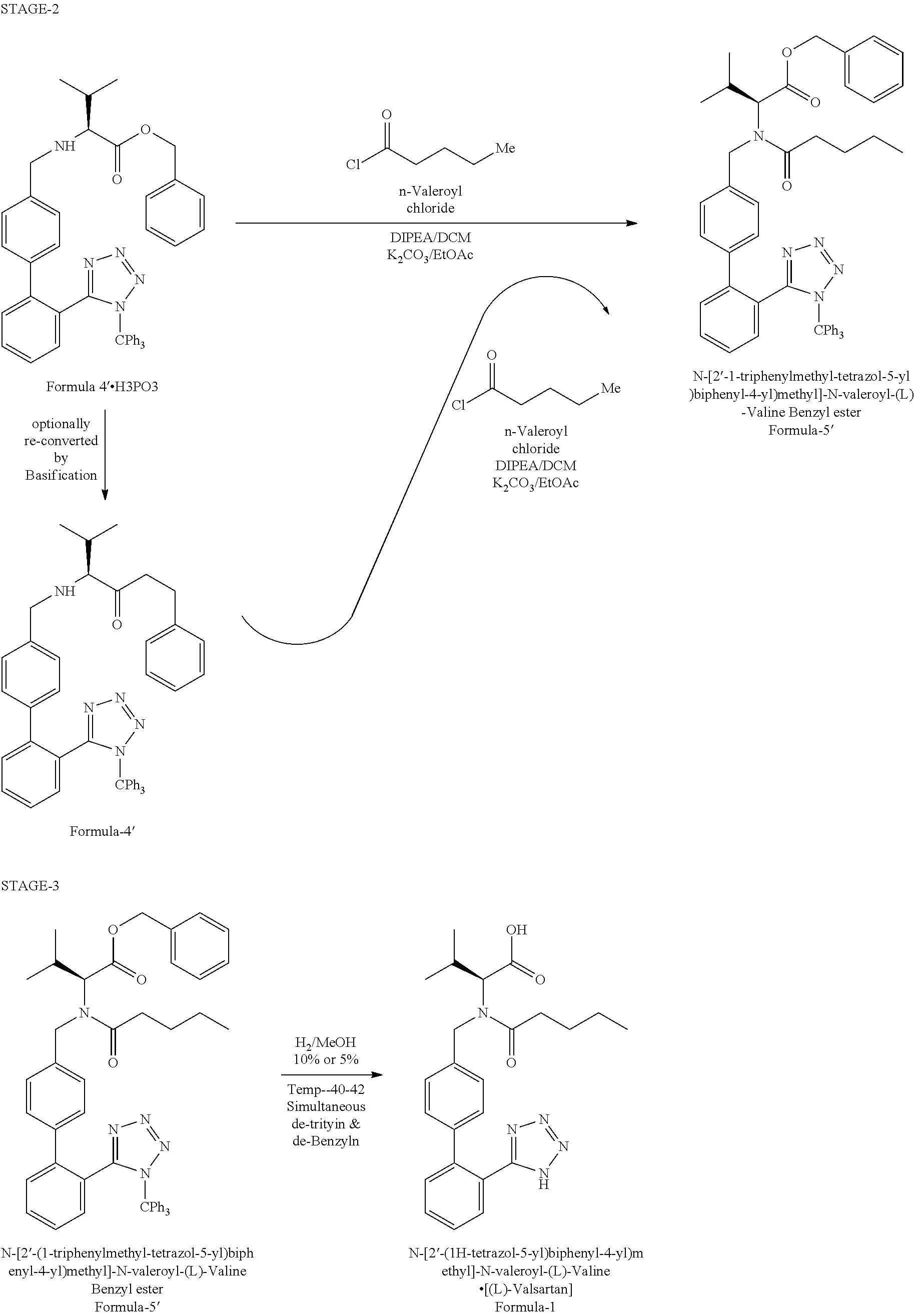

In accordance with the above objective, the present invention provides a novel phosphorous acid addition salt of formula 4

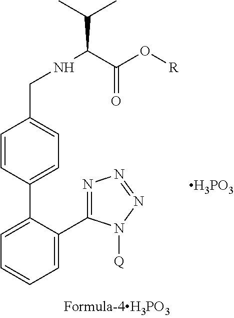

where Q is H or a N-protecting group, R is a carboxylic acid protecting group selected from substituted or unsubstituted C₁-C₆ alkyl group, substituted or unsubstituted aryl group, aralkyl group and the like which is suitable as a key intermediate for the synthesis of valsartan of high purity and yield.

Another object of the present invention is to provide a process for the synthesis of valsartan of high purity via isolation of a key intermediate N-[(2′-(1-triphenyl methyl tetrazole-5-yl)bi-phenyl]-4-yl]methyl]L-valine benzyl ester as a phosphite salt of Formula 4′.H₃PO₃

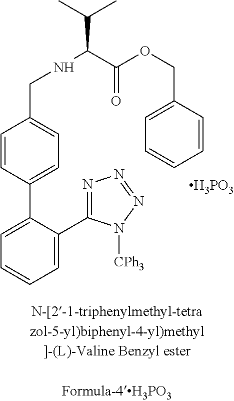

Yet another aspect of the present invention is to provide a method of purification of the key intermediate

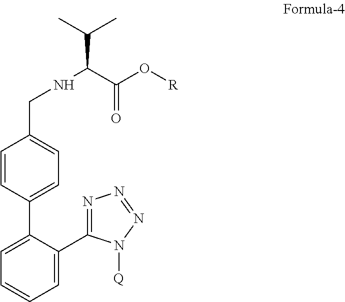

where Q is H or a N-protecting group, R is a carboxylic acid protecting group selected from substituted or unsubstituted C₁-C₆ alkyl group, substituted or unsubstituted aryl group, aralkyl group and the like via its phosphorous acid addition salt.

A preferred aspect of the present invention is to provide a method of purification of the key intermediate

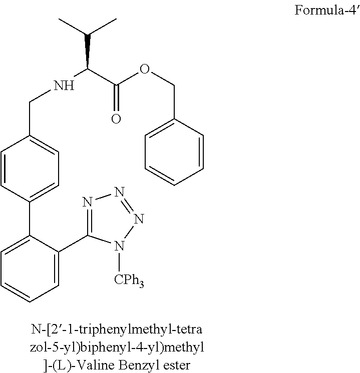

via its phosphorous acid addition salt.

Still another objective of the present invention is to provide a novel process for the N-alkylation of L-valine ester with a compound of Formula 3 or 3′ in the absence of a base.

Yet another objective of the present invention is to provide a single one-pot process of de-protection of a tritylated benzyl valsartan of formula

[symbol]

by a simple hydrogenation step involving simultaneous de-ation.

t invention can be summarized in the following steps: [symbol] paration of N-(1-oxopentyl)-N-[[2′-(1H-tetrazol-5-yl)(1,1′-biphenyl-4- [symbol] 1, [symbol] met

which comprises: [symbol] ormula 3 [symbol] is a halogen and Q is H or a Nitrogen protecting Group with a L-valine

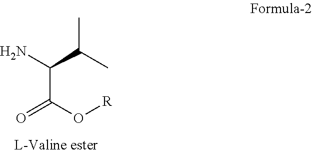

where R is a carboxylic acid protecting group selected from substituted or unsubstituted C₁-C₆ alkyl group, substituted or unsubstituted aryl group, aralkyl group and the like to afford a compound of formula-4

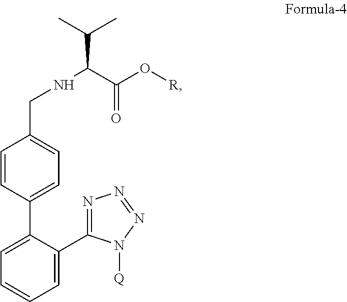

ii. converting the compound of formula-4 to its phosphite salt of Formula-4.H₃PO₃

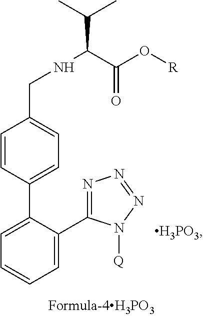

iii. preparing a compound of Formula-5

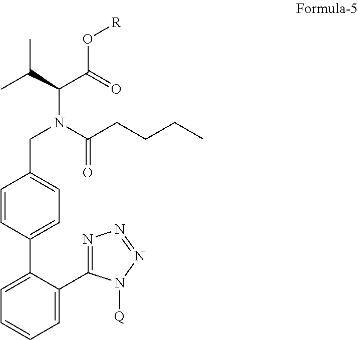

by

a) optionally treating the compound of formula-4.H₃PO₃ with a base in a solvent followed by reacting with n-valeroyl chloride in the presence of a base in a solvent to produce a compound of formula-5, OR

b) directly reacting a compound of formula-4.H₃PO₃ with n-valeroyl chloride in the presence of a base in a solvent to produce N-[2′-1-protected-tetrazol-5-yl)biphenyl-4-yl)methyl]-N-valeroyl-(L)-valine ester of formula-5,

iv. de-protecting the compound of Formula-5 to afford N-(1-oxopentyl)-N-[[2′-(1H-tetrazol-5-yl) (1,1′-biphenyl-4-yl]methyl]-L-valine of Formula 1.

B. The process according to A (i) above, wherein the reaction is carried out in the absence of a base.

C. The process according to A above wherein the nitrogen protecting group in compounds of Formula-3, Formula-4 and Formula-4.H₃PO₃ is preferably a triphenyl methyl group.

D. The process according to A above wherein the carboxylic acid protecting group in compounds of Formula-2, Formula-4 and Formula-4.H₃PO₃ is preferably a benzyl group.

E. A process for the preparation of N-(1-oxopentyl)-N-[[2′-(1H-tetrazol-5-yl)(1,1′-biphenyl-4-yl]methyl]-L-valine of Formula 1, which comprises:

i. reacting a 4-halomethyl 2′-(1-triphenyl methyl-tetrazol-5-yl)biphenyl compound of Formula 3′

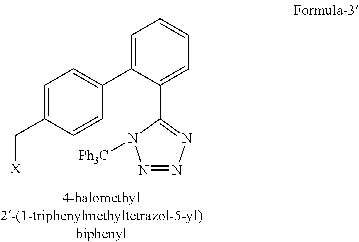

with a L-valine benzyl ester of Formula-2′

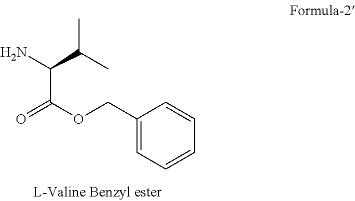

to afford N-[2′-triphenylmethyl-tetrazol-5-yl) biphenyl-4-yl)methyl]-(L)-valine benzyl ester of formula-4′ [symbol] [symbol] yl-tetra) methyl Aar [symbol] npound of formula-4′ to its Phosphite salt of Formula-4′.H₃PO₃ salt

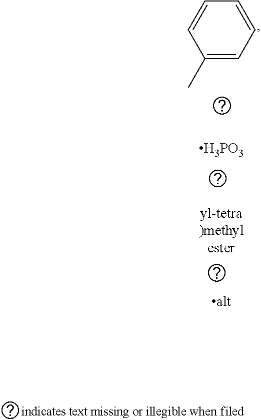

N-[2′-1-triphenylmethyl-tetrazol-5-yl)biphenyl-4-yl)methyl]-N-valeroyl-(L)-valine [symbol] nula-5′

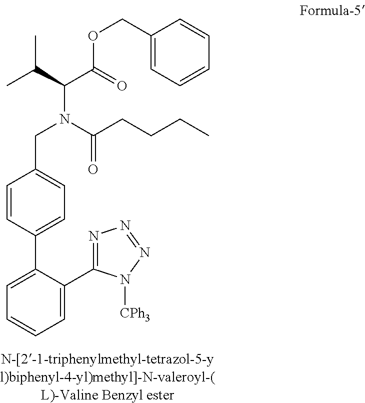

by a. optionally treating the compound of formula-4′.H₃PO₃ salt with a base in a solvent followed by reacting with n-valeroyl chloride in the presence of a base in a solvent to produce N-[2′-1-triphenylmethyl tetrazol-5-yl)biphenyl-4-yl)methyl]-N-valeroyl-(L)-valine benzyl ester of formula-5′, OR

b. directly reacting a compound of formula-4′.H₃PO₃ salt with n-valeroyl chloride in the presence of a base in a solvent to produce N-[2′-1-triphenylmethyltetrazol-5-yl)biphenyl-4-yl)methyl]-N-valeroyl-(L)-valine benzyl ester of formula-5′,

iv. de-protecting the compound of Formula-5′ to afford N-(1-oxopentyl)-N-[[2′-(1H-tetrazol-5-yl) (1,1′-biphenyl-4-yl]methyl]-L-valine of Formula 1.

F. The process according to E (i) above, wherein the reaction is carried out in the absence of a base.

G. The process according to E (iv) above, wherein the de-protection of compound of formula 5′ to afford N-(1-oxopentyl)-N-[[2′-(1H-tetrazol-5-yl)(1,1′-biphenyl-4-yl]methyl]-L-valine of Formula 1 is a one-pot process comprising simultaneous de-benzylation and de-tritylation.

H. A process for the preparation of N-(1-oxopentyl)-N-[[2′-(1H-tetrazol-5-yl)(1,1′-biphenyl-4-yl]methyl]-L-valine of Formula 1, which comprises:

preparing a compound of formula-4

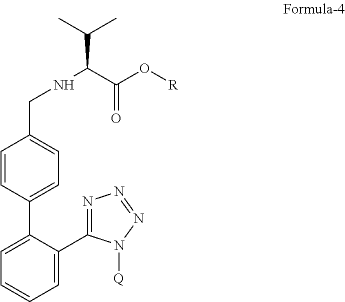

by reacting a compound of Formula 3

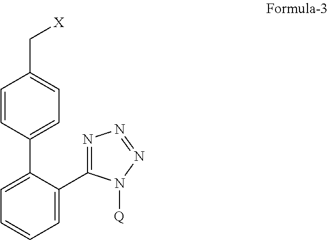

with a L-valine ester of Formula-2

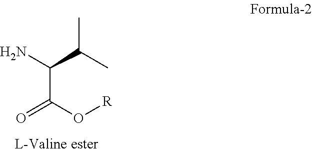

wherein X, R and Q have the same meanings as above in the absence of a base.

I. The process according to H above, wherein Q is preferably a triphenyl methyl group and R is a benzyl group.

J. A process for the preparation of N-(1-oxopentyl)-N-[[2′-(1H-tetrazol-5-yl)(1,1′-biphenyl-4-yl]methyl]-L-valine of Formula 1, which comprises de-protecting a compound of Formula-5′ to afford N-(1-oxopentyl)-N-[[2′-(1H-tetrazol-5-yl)(1,1′-biphenyl-4-yl]methyl]-L-valine of Formula 1 by a one-pot process comprising simultaneous de-benzylation and de-tritylation.

K. A compound of Formula

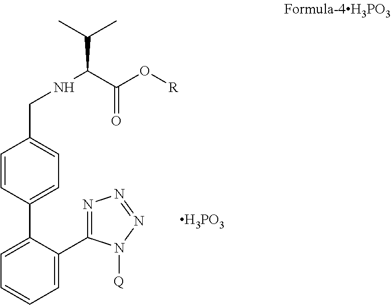

and hydrates or solvates thereof where X, R and Q have meanings as described above.

L. A compound of Formula

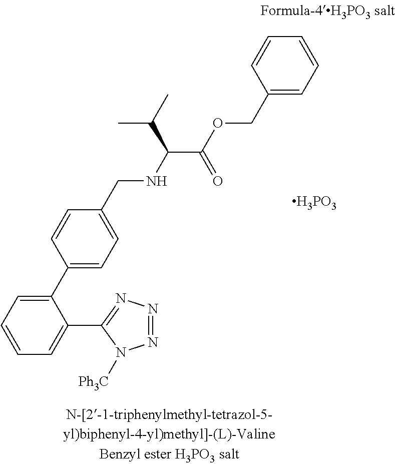

and hydrates or solvates thereof.

### BRIEF DESCRIPTION OF DRAWING FIGURES

Further objects of the present invention together with additional features contributing thereto and advantages accruing there from will be apparent from the following description of preferred.

Scheme: 1 is a schematic representation of a prior art process for the preparation of valsartan as taught by U.S. Pat. No. 5,399,578 starting from the reaction of 4-bromomethyl-2′-cyanobiphenyl (II) with L-valine methyl ester.

Scheme: 2 is a schematic representation of a prior art process for the preparation of valsartan as taught by U.S. Pat. No. 5,399,578 starting from the reaction of 4-bromomethyl-2′-(1-triphenylmethyltetrazol-5-yl) biphenyl (VI) with L-valine benzyl ester.

Scheme: 3 is a schematic representation of an improvised process of the present invention for the preparation of Valsartan of Formula (1).

Scheme: 4 is a schematic representation of a preferred embodiment of the present invention for the preparation of Valsartan of Formula (1).

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The objective of the present invention is to provide an improved process for the preparation of angiotensin II antagonists and intermediates thereof. Representative examples of angiotensin receptor antagonists include biphenyl compounds of the like of Valsartan, Losartan, Irbesartan and Candesartan. Particularly, the invention relates to an improved process for the preparation of valsartan (I) and its pharmaceutically acceptable salts or intermediates thereof.

The process of the present invention is described in Scheme-3 above. Scheme-4 above illustrates a preferred aspect of the present invention.

In accordance with the above objective, the present invention provides a novel Phosphorous acid addition salt of formula 4

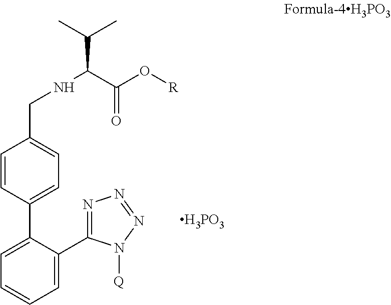

where Q is H or a N-protecting group, R is a carboxylic acid protecting group selected from substituted or unsubstituted C₁-C₆ alkyl group, substituted or unsubstituted aryl group, aralkyl group and the like via′ its phosphorous acid addition salt which is suitable as a key intermediate for the synthesis of valsartan of high purity and yield.

Another object of the present invention is to provide a process for the synthesis of valsartan of high purity via isolation of a key intermediate N-[[2′-(1-triphenyl methyl tetrazole-5-yl)bi-phenyl]-4-yl]methyl]L-valine benzyl ester as a phosphite salt of Formula 4′.H₃PO₃

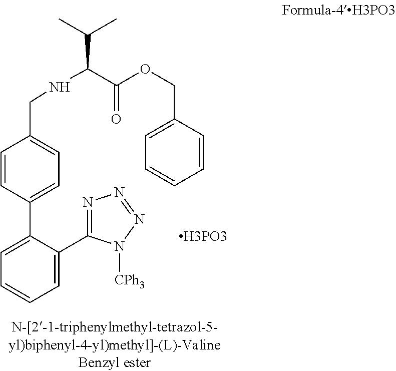

Yet another aspect of the present invention is to provide a method of purification of the key intermediate

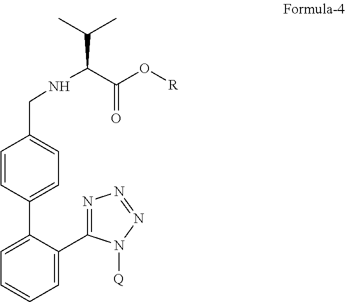

via its phosphorous acid addition salt.

A preferred aspect of the present invention is to provide a method of purification of the key intermediate

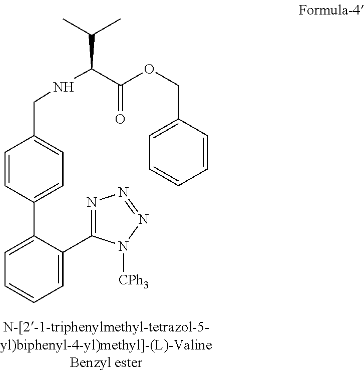

via its phosphorous acid addition salt.

Still another objective of the present invention is to provide a novel process for the N-alkylation of L-valine ester with 4-halomethyl 2′-(1-protected-tetrazol-5-yl)bi-phenyl compound in the absence of a base.

Yet another objective of the present invention is to provide a single one-pot process of de-protection of a tritylated benzyl valsartan of formula

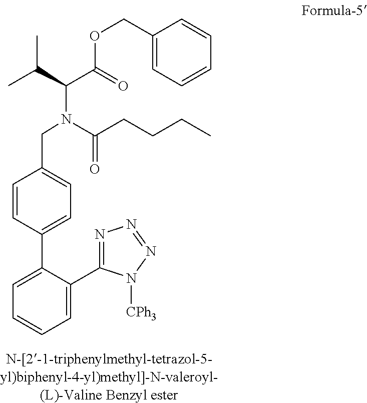

by a simple hydrogenation step involving simultaneous de-benzylation as well as de-tritylation.

Thus the present invention provides an improved process for the preparation of valsartan which comprises

i. reacting a compound of Formula 3

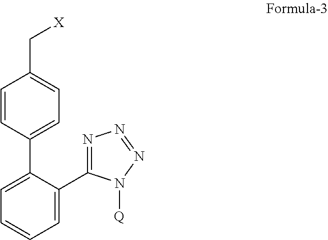

where X is a halogen and Q is a Nitrogen protecting Group with a L-valine ester of Formula-2

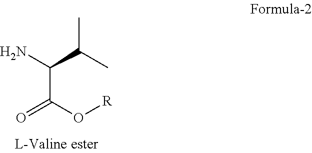

where R is a carboxylic acid protecting group selected from substituted or unsubstituted C₁-C₆ alkyl group, substituted or unsubstituted aryl group, aralkyl group and the like to afford a compound of formula-4

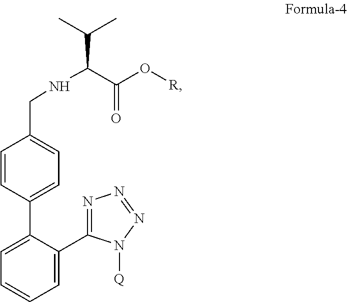

ii. converting the compound of formula-4 to its Phosphite salt of Formula-4.H₃PO₃ salt

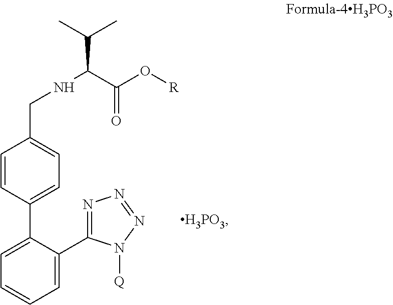

iii. preparing compound of Formula-5

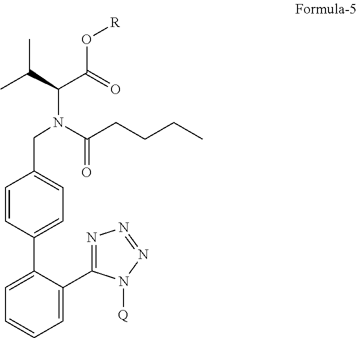

by

a) optionally treating the compound of formula-4.H₃PO₃ salt with a base in a solvent followed by reacting with n-valeroyl chloride in the presence of a base in a solvent to produce a compound of formula-5, OR

b) directly reacting a compound of formula-4.H₃PO₃ salt with n-valeroyl chloride in the presence of a base in a solvent to produce a compound of formula-5,

iv. de-protecting the compound of Formula-5 to afford N-(1-oxopentyl)-N-[[2′-(1H-tetrazol-5-yl)(1,1′-biphenyl-4-yl]methyl]-valine of Formula I.

In a preferred embodiment of the present invention, the N-alkylation of L-valine ester

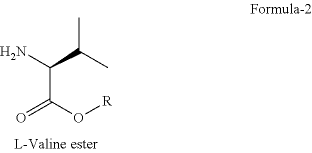

with 4-halomethyl 2′-(1-triphenyl methyl-tetrazol-5-yl) biphenyl compound is carried out in the absence of a base.

The suitable protecting groups for protected 5-tetrazolyl are those customarily used in tetrazole chemistry, in particular triphenylmethyl, benzyl which is unsubstituted, for example by nitro, such as 4-nitrobenzyl, lower alkoxymethyl, such as methoxy and ethoxymethyl, lower alkylthiomethyl, such as methylthiomethyl, silyl, such as tri-lower alkyl silyl, for example dimethyl ter-butyl- and triisopropylsilyl, and 2-cyanoethyl, also lower alkoxy-lower alkoxymethyl, such as 2-methoxyethoxymethyl, benzyloxymethyl and phenacyl. A preferred embodiment of the present invention employs triphenylmethyl protecting group.

The solvents selected for the reaction may be those that are well known in the art such as protic and aprotic solvents, alcohols, esters, nitriles, ethers, aliphatic and aromatic hydrocarbons, amides, ketones and the like or mixtures thereof. For example, the solvent may be an inert polar aprotic, preferably anhydrous, solvent or solvent mixture, for example in a carboxamide, for example formamide or dimethylformamide, a halogenated hydrocarbon, for example methylene chloride, carbontetrachloride or chlorobenzene, a ketone, for example acetone, cyclic ethers, for example tetrahydrocarbon, an ester, for example, ethylacetate, or a nitrile, for example, acetonitrile, or in mixtures thereof, if desired at reduced or elevated temperature, for example in a temperature range between ambient temperature and the boiling point of the solvent. The term ambient temperature in the present application is intended to indicate the temperature usually found in industrial laboratory and production facilities such as in the range of 15-30° C., preferably in the range of 20-25° C.

In a preferred embodiment of the process of the present invention (L)-valine benzyl ester was reacted with 4-bromomethyl-2′-(1-triphenylmethyl-tetrazol-5-yl) biphenyl in the absence of a base in Ethyl acetate at 55-60° C. and the progress of the reaction monitored by TLC and HPLC. To the Ethylacetate layer was added phosphorous acid and stirred for around 1 hour. The phosphorous acid addition salt of N-[2′-1-triphenylmethyl-tetrazol-5-yl)biphenyl-4-yl)methyl]-(L)-valine benzyl ester was isolated as solid, was filtered, dried in vacuum and was found to have a purity as high as 97% by HPLC.

The N-acylation reactions may be performed by conventional techniques apparent to a person skilled in the art. The stage-1 phosphorous addition salt of stage-1 may either be re-converted back to its free base by conventional way of extraction in an organic solvent and aqueous base treatment or directly coupled with the acylating agent in the presence of excess base using conventional N-acylation techniques apparent to a person skilled in the art.

In an embodiment of the present invention, the stage-1 phosphite salt was dissolved in distilled water and the pH adjusted to about 7-8 and extracted with DCM. To the combined DCM extracts was added Diisopropylamine, n-valeroyl chloride at about −5° C. to −10° C. and the progress of the reaction monitored by HPLC. The reaction mixture was worked up by standard procedures comprising bicarbonate washes, HCl washes, water washes and finally the solvent stripped under vacuum to afford the N-acylated product.

The de-protection of compounds of formula-5 may be carried out by conventional techniques apparent to a person skilled in the art such as by de-esterification for removal of ester group and de-tritylation for removal of a trityl group by acidic or basic hydrolysis.

A preferred embodiment of the present invention provides a single one-pot process of de-protection of a tritylated benzyl valsartan of formula

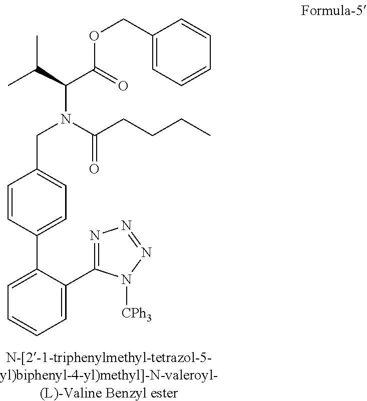

by a simple hydrogenation step involving simultaneous de-benzylation as well as de-tritylation. In an embodiment of the present invention, a one-pot de-benzylation and de-tritylation was carried out in an autoclave in the presence of 10% Pd—C at about 3-5 kg/cm² in the presence of a base such as diisopropylethylamine in methanol.

The following examples are intended to illustrate the scope of the present invention in all its aspects but not to limit it thereto.

### EXAMPLES

### Example 1

### Stage-1—Preparation of N-[2′-1-triphenylmethyl-tetrazol-5-yl)biphenyl-4-yl)methyl]-(L)-valine benzyl ester.H₃PO₃ salt

104.6 g L-Valine benzyl ester hydrochloride was dissolved in DM-water and basified with 20% sodium hydroxide solution to pH of about 10-12. Ethyl acetate was added and stirred for 20 min and the ethyl acetate layer was separated. To the ethyl acetate layer was added TTBB and stirred for 12-14 hrs at 55-60° C. The Ethyl acetate layer was washed with DM water.

The aqueous layer was preserved for the recovery of L-Valine benzyl ester. Phosphorous acid (23.2 g) was added to the ethyl acetate layer under stirring and stirred for 1.0 hr

The solids were filtered and stirred in DM-water for 30 min, filtered, suck dried well and dried in vacuum over at 55-60° C. till moisture content was NMT 2.0%

Weight: 125-130 g % Purity (by HPLC): 97.0%(+) Yield: 91-95%

### Example 2

### Stage-2—Preparation of N-[2′-1-triphenylmethyl-tetrazol-5-yl)biphenyl-4-yl)methyl]-N-valeroyl-(L)-valine benzyl ester

Stage-I phosphite salt (100 g) from example 1 above was dissolved in DM-water (500 ml) and the pH of aqueous layer adjusted to 7-8 and extracted with dichloromethane (250×2)

To the extracted dichloromethane was added Diisopropyl ethyl amine (25.3 g) under stirring at −5 to −10° C. After addition, the reaction mixture was stirred for 30 min at −5 to −10° C. N-Valeroyl chloride (24.2 g by diluting with 200 ml dichloromethane) was added slowly under stirring for 1-2 hrs maintaining pot temperature between −5 to −10° C. After addition, the reaction mixture was stirred for 2-3 hrs and monitored for completion by HPLC. The reaction mass washed with 5% NaHCO₃ solution, 5% HCl solution and followed by 5% NaHCO₃ solution and finally the solvent was stripped under vacuum to provide the title stage-2 intermediate.

Weight: 115-120 g % Purity: 97.0%

### Example 3

### Stage-3—Preparation of (L)-Valsartan

Stage-II (100 g) was dissolved in 1.0 lt methanol and charged into the autoclave About 2-3 ml DIPEA (Diisopropyl ethyl amine) was added, then 10 g 10% Pd/C (50% wet) and pressure of 3-5 kg/cm² maintained for 6.0 hrs and the reaction mixture monitored by HPLC. The side product was isolated by filtration, the filtrate was concentrated completely and dissolved in 5% caustic solution, washed with Toluene (300 ml×3), acidified with 5% HCl solution and pH adjusted to 2-3. The solids were extracted with EtOAc (150 ml×2), the solvent stripped completely.

Weight: 100-110 g % Purity (by HPLC): 97.0% (+)

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are preferred by way of example to facilitate comprehension of the invention and should not be construed to limit the scope thereof.

1. A process for the preparation of N-(1-oxopentyl)-N-[[2′-(1H-tetrazol-5-yl)(1,1′-biphenyl-4-yl]methyl]-L-valine of Formula 1, 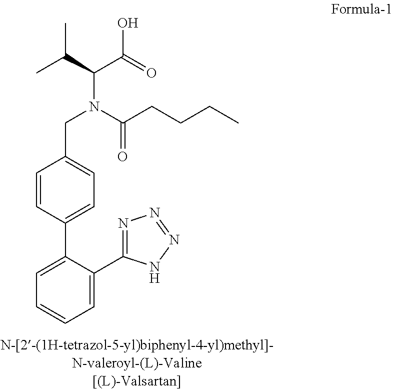 [SMILES: CCCCC(=O)N(Cc1ccc(-c2ccccc2-c2nnn[nH]2)cc1)[C@H](C(=O)O)C(C)C]
which comprises:
i. reacting a 4-halomethyl 2′-(1-protected-tetrazol-5-yl)biphenyl compound of Formula 3 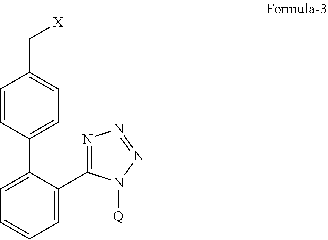
where X is a halogen and Q is H or a Nitrogen protecting Group
with a L-valine ester of Formula-2 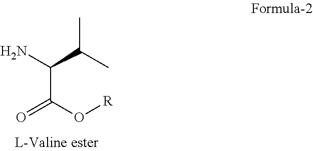
where R is a carboxylic acid protecting group selected from substituted or unsubstituted C₁-C₆ alkyl group, substituted or unsubstituted aryl group, aralkyl group and the like
to afford a compound of formula-4 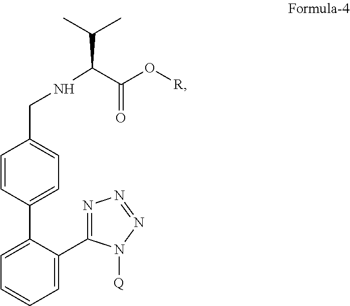
ii. converting the compound of formula-4 to its phosphite salt of Formula-4.H₃PO₃ 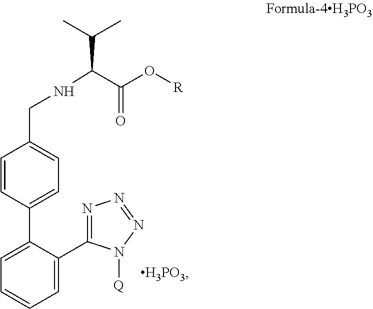
iii. preparing a compound of Formula-5 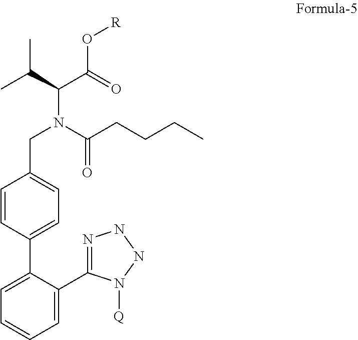
by
a) optionally treating the compound of formula-4.H₃PO₃ with a base in a solvent followed by reacting with n-valeroyl chloride in the presence of a base in a solvent to produce a compound of formula-5, OR
b) directly reacting a compound of formula-4.H₃PO₃ with n-valeroyl chloride in the presence of a base in a solvent to produce N-[2′-1-protected-tetrazol-5-yl)biphenyl-4-yl)methyl]-N-valeroyl-(L)-valine ester of formula-5,
iv. de-protecting the compound of Formula-5 to afford N-(1-oxopentyl)-N-[[2′-(1H-tetrazol-5-yl) (1,1′-biphenyl-4-yl]methyl]-L-valine of Formula 1.

2. The process according to claim 1, wherein the reaction is carried out in the absence of a base.

3. The process according to claim 1, wherein the nitrogen protecting group in compounds of Formula-3, Formula-4 and Formula-4.H₃PO₃ is preferably a triphenyl methyl group.

4. The process according to claim 1, wherein the carboxylic acid protecting group in compounds of Formula-2, Formula-4 and Formula-4.H₃PO₃ is preferably a benzyl group.

5. A process for the preparation of N-(1-oxopentyl)-N-[[2′-(1H-tetrazol-5-yl)(1,1′-biphenyl-4-yl]methyl]-L-valine of Formula 1, which comprises:
i. reacting a 4-halomethyl 2′-(1-triphenyl methyl-tetrazol-5-yl)biphenyl compound of Formula 3′ 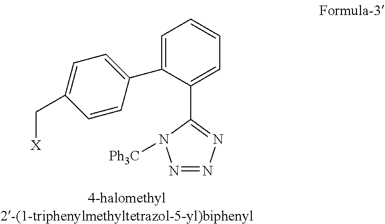
with a L-valine benzyl ester of Formula-2′ 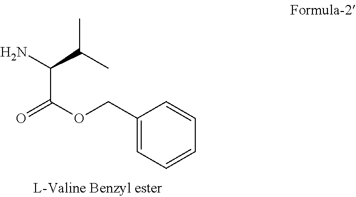
to afford N-[2′-triphenylmethyl-tetrazol-5-yl) biphenyl-4-yl)methyl]-(L)-valine benzyl ester of formula-4′ 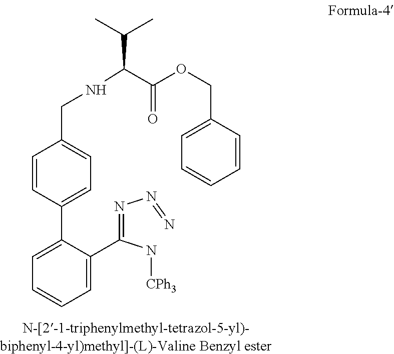
ii. converting the compound of formula-4′ to its Phosphite salt of Formula-4′.H₃PO₃ salt 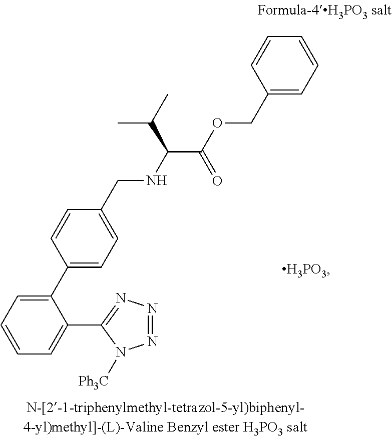
iii. preparing N-[2′-1-triphenylmethyl-tetrazol-5-yl)biphenyl-4-yl)methyl]-N-valeroyl-(L)-valine benzyl ester of Formula-5′ 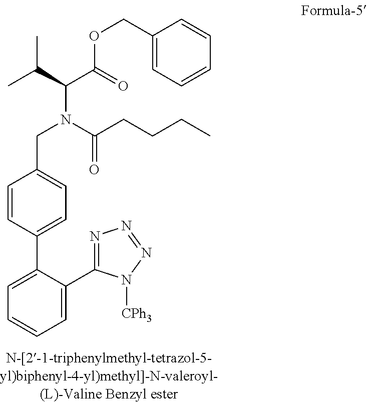
by
a. optionally treating the compound of formula-4′.H₃PO₃ salt with a base in a solvent followed by reacting with n-valeroyl chloride in the presence of a base in a solvent to produce N-[2′-1-triphenylmethyl tetrazol-5-yl)biphenyl-4-yl)methyl]-N-valeroyl-(L)-valine benzyl ester of formula-5′, OR
b. directly reacting a compound of formula-4.H₃PO₃ salt with n-valeroyl chloride in the presence of a base in a solvent to produce N-[2′-1-triphenylmethyltetrazol-5-yl)biphenyl-4-yl)methyl]-N-valeroyl-(L)-valine benzyl ester of formula-5′,
iv. de-protecting the compound of Formula-5′ to afford N-(1-oxopentyl)-N-[[2′-(1H-tetrazol-5-yl) (1,1′-biphenyl-4-yl]methyl]-L-valine of Formula 1.

6. The process according to claim 5, wherein the reaction is carried out in the absence of a base.

7. The process according to claim 5, wherein the de-protection of compound of formula 5′ to afford N-(1-oxopentyl)-N-[[2′-(1H-tetrazol-5-yl) (1,1′-biphenyl-4-yl]methyl]-L-valine of Formula 1. is a one-pot process comprising simultaneous de-benzylation and de-tritylation.

8. A process for the preparation of N-(1-oxopentyl)-N-[[2′-(1H-tetrazol-5-yl)(1,1′-biphenyl-4-yl]methyl]-L-valine of Formula 1, which comprises:
preparing N-[2′-1-protected-tetrazol-5-yl) biphenyl-4-yl)methyl]-(L)-valine ester of formula-4 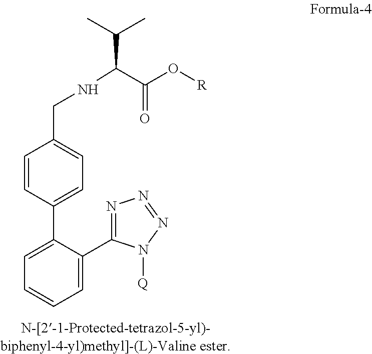
by reacting a 4-halomethyl 2′-(1-protected-tetrazol-5-yl)biphenyl compound of Formula 3′ 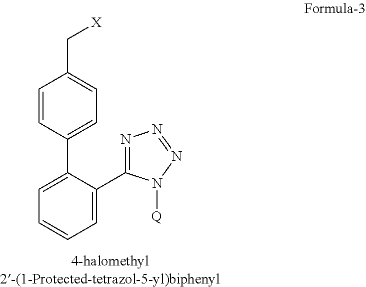
with a L-valine ester of Formula-2′ 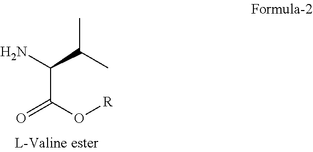
wherein X, R and Q have the same meanings as above in the absence of a base.

9. The process according to claim 8, wherein Q is preferably a triphenyl methyl group and R is a benzyl group.

10. (canceled)

11. (canceled)

12. (canceled)
